(19) United States
(12) Patent Application Publication
(10) Pub. No.: US 2026/0035656 A1
(43) Pub. Date: Feb. 5, 2026

(54) STRAIN OF LACTIC-ACID-STRESS-TOLERANT ZYGOSACCHAROMYCES BAILII CAPABLE OF ACHIEVING HIGH-YIELD PRODUCTION OF ETHANOL AND FLAVOR COMPOUNDS

(71) Applicant: Jiangnan University, Wuxi (CN)

(72) Inventors: Hai DU, Wuxi (CN); Yan XU, Wuxi (CN); Junlin WEI, Wuxi (CN)

(21) Appl. No.: 19/354,071

(22) Filed: Oct. 9, 2025

Related U.S. Application Data
(63) Continuation of application No. PCT/CN2024/110474, filed on Aug. 7, 2024.

(51) Int. Cl. C12N 1/16; A23C 9/12; A23C 11/10; A23L 2/02; A23L 2/38; A23L 27/50; C12G 3/021; C12G 3/022; C12J 1/00; C12R 1/645

(52) U.S. Cl. CPC C12N 1/165; A23C 9/1203; A23C 11/106; A23L 2/02; A23L 2/382; A23L 27/50; C12G 3/021; C12G 3/022; C12J 1/00; C12R 2001/645

(57) ABSTRACT

The present invention belongs to the technical field of microorganisms. Disclosed is a strain of lactic-acid-stress-tolerant Zygosaccharomyces bailii capable of achieving high-yield production of ethanol and flavor compounds. The Zygosaccharomyces bailii CCTCC NO: M 2023729 of the present invention is screened from Chinese Maotai-flavor liquor yeast, and the strain is not only tolerant of lactic acid stress, but also has lactic acid degrading capability, and can also achieve high-yield production of ethanol and various flavor compounds under the stress of lactic acid. The strain of the present invention can be prepared into a microbial agent for use in the fermentation production process of Maotai-flavor liquor, and can significantly increase the content of ethanol and flavor compounds in fermented grain.

### TECHNICAL FIELD

The present invention belongs to the technical field of microorganisms, and relates to a strain of lactic-acid-stress-tolerant Zygosaccharomyces bailii capable of achieving high-yield production of ethanol and flavor compounds.

### BACKGROUND

During the brewing process of traditional fermented food, microorganisms are subjected to various environmental stresses, such as high acid, high ethanol, and high temperatures. Lactic acid is a metabolic by-product of microorganisms commonly found in traditional fermented foods. On the one hand, lactic acid contributes to the rich and mellow mouthfeel of fermented foods; on the other hand, due to its low volatility and other characteristics, lactic acid tends to accumulate over time during the brewing process, resulting in increased acidity in a fermentation system and inhibiting the growth and metabolism of functional microorganisms. For example, in the brewing process of Maotai-flavor liquor that involves repeated fermentation in multiple batches, the unique fermentation process leads to accumulative lactic acid across fermentation batches, making a lactic acid content reach as high as 40 g/kg. In low-yield fermentation batches, the lactic acid content may be even higher, significantly exceeding a normal concentration range of the liquor, resulting in a reduction or even loss of desirable flavor compounds.

The accumulation of lactic acid gives rise to problems such as low yield and flavor defects during fermentation. However, due to the characteristics of open and naturally inoculated fermentation, these problems cannot be effectively solved by simply changing the operating process. An effective method is to isolate and culture strains with excellent traits, and apply them to the low-yield fermentation batches. Nevertheless, most current research focuses primarily on analyzing the lactic acid tolerance characteristics of specific functional microorganisms. However, fermented foods are sensory products, with flavor compounds as quantitative indicators for their product quality. These functional microorganisms must not only tolerate a highly acidic environment during the fermentation process but also actively contribute to the formation of flavor compounds. Therefore, strains selected for fermentation must have various traits, including lactic acid tolerance and rich flavor compounds, which poses a considerable challenge for strain screening.

### SUMMARY

The present invention provides a functional yeast strain Zygosaccharomyces bailii LBM888, which is isolated from a Maotai-flavor liquor brewing system. The strain is not only tolerant of lactic acid stress, but also has a lactic acid degrading capability to achieve high-yield production of ethanol and various flavor compounds under the stress of lactic acid. The strain of the present invention can be prepared into a microbial agent for use in a fermentation production process of Maotai-flavor liquor, and can significantly increase an ethanol content and flavor compounds in fermented grain. The strain of the present invention has excellent performance in practical application and can be applied to the actual production of fermenting foods.

The present invention provides a strain of Zygosaccharomyces bailii LBM888. The Zygosaccharomyces bailii LBM888 was deposited with China Center for Type Culture Collection on May 10, 2023, with a deposit address of Wuhan University, Wuhan City, P. R. China, and an accession number of CCTCC NO: M 2023729.

The Zygosaccharomyces bailii CCTCC NO: M 2023729 has the following characteristics:
(1) Morphological characteristics: colony morphology of the strain on a YPD plate is white and opaque with a certain gloss; and the colony has regular, smooth, and circular edges with a bulge in a center. Under the transmission electron microscopy, a single cell is relatively large and oval-shaped, without flagella or mycelia;
(2) Growth characteristics: the strain reaches a stationary phase after 60 hours of growth in the sorghum juice medium, with an OD₆₀₀ of 3.36±0.05 at the end of fermentation; and it can grow under lactic acid stress in a fermentation medium containing 40 g/L lactic acid, with an OD₆₀₀ of 3.02±0.08;
(3) Carbon source utilization characteristics: The strain can utilize glucose, but cannot utilize glycerol, erythritol, xylitol, arabinitol, ribitol, sorbitol, mannitol, maltitol, xylose, D-ribose, D-arabinose, L-sorbose, L-rhamnose, D-galactose, D-trehalose, sucrose, stachyose, D-raffinose, D-melibiose, D-melezitose, maltotriose, maltose, gentiobiose, D-cellobiose, or dextrin;
(4) Ethanol production characteristics: After 60 hours of fermentation in a sorghum juice medium, glucose decreases from an initial 40 g/L to 4.09±0.23 g/L at the end of fermentation, with an ethanol yield of 13.66±0.45 g/L; and in a fermentation medium containing 40 g/L lactic acid, an ethanol contents reaches 11.99±0.13 g/L at the end of fermentation;
(5) Lactic acid tolerance and degradation: The strain can tolerate up to 40 g/L lactic acid; in a fermentation medium containing 40 g/L lactic acid, the strain sustains robust growth with an OD₆₀₀ of 3.02±0.08; a lactic acid content at the beginning of fermentation is 40 g/L, while 5.88 g/L is degraded at the end of fermentation; and the strain can produce a higher content of ethanol under lactic acid stress, and an ethanol content reaches 11.99±0.13 g/L at the end of fermentation;
(6) Production of flavor compounds under lactic acid stress: Flavor compounds with a content exceeding 0.1 g/L include ethyl acetate (1.02 g/L), isoamyl alcohol (1.52 g/L), phenylethyl alcohol (0.72 g/L), phenylethyl acetate (0.50 g/L), isobutanol (0.13 g/L), isoamyl lactate (0.10 g/L), and isovaleric acid (0.10 g/L). Flavor compounds with a content below 0.1 g/L include isoamyl acetate, ethyl lactate, and 1-octanol; and
(7) Application in Baijiu brewing: The strain can significantly reduce the lactic acid content, and increase the ethanol content and volatile flavor compounds in fermented grain.

A second objective of the present invention is to provide a microbial agent that contains the Zygosaccharomyces bailii strain CCTCC NO: M 2023729 of the present invention.

In one embodiment of the present invention, the microbial agent uses the Zygosaccharomyces bailii CCTCC NO: M 2023729 as a principal microorganism.

In one embodiment of the present invention, a viable count of the Zygosaccharomyces bailii CCTCC NO: M 2023729 in the microbial agent is 10⁵-10⁸ CFU/mL.

In one embodiment of the present invention, a microbial concentration of the Zygosaccharomyces bailii CCTCC NO: M 2023729 has a range of 10⁵-10⁸ CFU/mL.

In one embodiment of the present invention, the microbial agent is prepared by culturing a seed culture of the Zygosaccharomyces bailii CCTCC NO: M 2023729, followed by scale-up fermentation.

In one embodiment of the present invention, the microbial agent is a liquid microbial agent or a solid microbial agent containing the Zygosaccharomyces bailii CCTCC NO: M 2023729 of the present invention.

In one embodiment of the present invention, the microbial agent includes viable cells of the Zygosaccharomyces bailii CCTCC NO: M 2023729 strain of the present invention, dried cells of the Zygosaccharomyces bailii CCTCC NO: M 2023729 strain of the present invention obtained by freeze drying, and immobilized cells of the Zygosaccharomyces bailii CCTCC NO: M 2023729 strain of the present invention, or the Zygosaccharomyces bailii CCTCC NO: M 2023729 strain of the present invention in any other applicable form.

In one embodiment of the present invention, the microbial agent also contains any strain of genus capable of fermenting foods or the preparation of fermented foods, such as Bacillus licheniformis, Saccharomyces cerevisiae, and Bacillus subtilis.

In one embodiment of the present invention, the microbial agent also contains any carrier capable of fermenting foods.

A third objective of the present invention is to provide application of the Zygosaccharomyces bailii CCTCC NO: M 2023729 or a microbial agent containing the Zygosaccharomyces bailii CCTCC NO: M 2023729. The application includes one or more of the following: degradation of lactic acid, increase in an ethanol content, and increase in contents of flavor compounds. Specific application is as follows: a single colony of Zygosaccharomyces bailii CCTCC NO: M 2023729 is inoculated into a sorghum juice medium to obtain a seed culture after incubation; the seed culture of Zygosaccharomyces bailii CCTCC NO: M 2023729 is then inoculated into a fermentation medium added with lactic acid for fermentation; the sorghum juice medium is prepared by soaking sorghum in water at a ratio of 1:4 (M:V) overnight, amylase is then added for liquefaction, glucoamylase is added for saccharification at 60° C., filtration and centrifugation are finally performed, and a sugar content is adjusted to 5° Bx. The fermentation medium is prepared by adjusting a pH value of the sorghum juice medium to 3.2 using lactic acid.

A fourth objective of the present invention is to provide application of the Zygosaccharomyces bailii CCTCC NO: M 2023729 or a microbial agent containing the Zygosaccharomyces bailii CCTCC NO: M 2023729 in fermenting foods. The application includes adding the Zygosaccharomyces bailii CCTCC NO: M 2023729 or a microbial agent containing the Zygosaccharomyces bailii CCTCC NO: M 2023729 in the process of preparing the fermented foods.

In one embodiment of the present invention, the application is used in Baijiu brewing.

The present invention further provides a method for producing ethanol and flavor compounds under lactic acid stress. The method includes: selecting and inoculating a single colony of the Zygosaccharomyces bailii CCTCC NO: M 2023729 into sorghum juice medium to obtain a seed culture, and inoculating the seed culture of the Zygosaccharomyces bailii CCTCC NO: M 2023729 into a fermentation medium added with lactic acid for fermentation.

In one embodiment of the present invention, the sorghum juice medium is prepared by soaking sorghum with water at a 1:4 (M:V) ratio overnight, amylase is added for liquefaction by steaming, saccharification is performed with glucoamylase at 60° C., and filtering and centrifugation are finally performed to adjust a sugar concentration to 5° Bx.

The present invention further provides a method for producing ethanol and flavor compounds under lactic acid stress, including selecting and inoculating a single colony of the Zygosaccharomyces bailii CCTCC NO: M 2023729 into sorghum juice medium, culturing at 30° C. and 200 rpm for 24 hours to obtain a seed culture, inoculating 1 mL of the seed culture of the Zygosaccharomyces bailii CCTCC NO: M 2023729 into 100 mL of a fermentation medium added with 40 g/L lactic acid, and culturing at 30° C. and 200 rpm for 72 hours.

The present invention further provides application of a yeast agent in Baijiu brewing. The application includes: inoculating Zygosaccharomyces bailii CCTCC NO: M 2023729 into a seed culture medium to obtain a seed culture; centrifuging the seed culture to remove a supernatant, and resuspending precipitate in water to obtain a yeast agent.

The yeast agent is applied to a fourth fermentation round of a Maotai-flavor liquor brewing process with steps as follows:
distilling, spreading and cooling the fermented grain, mixing the cooled fermented grain with Qu a Chinese fermentation starter, taking out the fermented grain, evenly spraying the yeast agent onto the fermented grain, and mixing thoroughly; loading the fermented grain into a bamboo basket and placing in a fermentation pile and a fermentation pit for in-situ fermentation.

The present invention further provides application of a yeast agent in Baijiu brewing, including: inoculating the Zygosaccharomyces bailli CCTCC NO: M 2023729 into a seed culture medium, and culturing at 30° C. and 200 rpm for 48 hours to obtain a seed culture of 1×10⁸ cells/mL; taking and centrifuging 10 mL of the seed culture at 10,000×g for 5 minutes to remove a supernatant, and resuspending precipitate in 1000 mL of sterile water to obtain a yeast suspension; and

the yeast suspension is applied to a fourth fermentation round of a Maotai-flavor liquor brewing process with specific operations as follows:
distilling, spreading and cooling the fermented grain, mixing the cooled fermented grain with Qu a Chinese fermentation starter, taking out 30 kg of the fermented grain, evenly spraying 1000 mL yeast suspension onto 30 kg of the fermented grain, and mixing thoroughly; loading the fermented grain into a bamboo basket, and placing in a fermentation pile and a fermentation pit for in-situ fermentation.

The present invention has the following beneficial effects:

The Zygosaccharomyces bailii CCTCC NO: M 2023729 of the present invention is screened from Chinese Maotai-flavor liquor yeast, and the strain is not only tolerant of lactic acid stress, but also has a lactic acid degrading capability to achieve high-yield production of ethanol and various flavor compounds under the stress of lactic acid. The strain of the present invention can be prepared into a microbial agent for use in the fermentation production process of Maotai-flavor liquor, and can significantly increase an ethanol content and flavor compounds in fermented grain. The strain of the present invention has excellent performance in practical application and can be applied to the actual production of fermenting foods.

### Deposit of Biological Materials

The Zygosaccharomyces bailii CCTCC NO: M 2023729 is taxonomically designated as Zygosaccharomyces bailii LBM888, and was deposited with China Center for Type Culture Collection (CCTCC) on May 10, 2023, with a deposit address of Wuhan University, Wuhan City, P. R. China, and an accession number of CCTCC NO: M 2023729.

### BRIEF DESCRIPTION OF THE DRAWINGS

FIG. 1A shows colony morphology of Zygosaccharomyces bailii CCTCC NO: M 2023729 on a YPD plate.

FIG. 1B shows morphology of Zygosaccharomyces bailii CCTCC NO: M 2023729 under transmission electron microscopy.

FIG. 2 shows a growth curve of Zygosaccharomyces bailii CCTCC NO: M 2023729 (n=3).

FIG. 3 shows carbon source utilization of Zygosaccharomyces bailii CCTCC NO: M 2023729.

FIG. 4 shows growth of Zygosaccharomyces bailii CCTCC NO: M 2023729 under lactic acid stress; “ZB-0” indicates no lactic acid added; “ZB+Lac” indicates stress with 40 g/L lactic acid; *, P<0.05; **, P<0.01 (Tukey's test).

FIG. 5A shows lactic acid degradation of Zygosaccharomyces bailii CCTCC NO: M 2023729 under lactic acid stress.

FIG. 5B shows ethanol production capacity of Zygosaccharomyces bailii CCTCC NO: M 2023729 under lactic acid stress.

FIG. 6 shows production of volatile flavor compounds of Zygosaccharomyces bailii CCTCC NO: M 2023729 under lactic acid stress.

FIG. 7 shows a flowchart of application of Zygosaccharomyces bailii CCTCC NO: M 2023729 in a fermentation process of Maotai-flavor liquor.

FIG. 8A shows effect of adding a microbial agent of Zygosaccharomyces bailii CCTCC NO: M 2023729 on lactic acid content in a fermentation process of Maotai-flavor liquor.

FIG. 8B shows effect of adding a microbial agent of Zygosaccharomyces bailii CCTCC NO: M 2023729 on ethanol content in a fermentation process of Maotai-flavor liquor.

FIG. 9 shows a production process flow diagram of Maotai-flavor liquor.

### DETAILED DESCRIPTIONS OF THE EMBODIMENTS

### (I) Culture Medium

Screening medium (YPD): 20 g/L glucose, 20 g/L peptone, 10 g/L yeast extract, and 20 g/L agar powder.

Seed medium: 20 g/L glucose, 20 g/L peptone, and 10 g/L yeast extract.

Sorghum juice medium: sorghum is soaked with water at a 1:4 (M:V) ratio overnight, amylase is added for liquefaction by steaming, saccharification is performed with glucoamylase at 60° C., and filtering and centrifugation are finally performed to adjust a sugar concentration to 5° Bx.

### (II) Method for Lactic Acid Content Determination

For a fermentation broth obtained from a simulation experiment, the fermentation broth is filtered with a 0.22 μm aqueous-phase membrane filter to obtain a filtrate, and the filtrate is transferred to a high-performance liquid chromatography (HPLC) vial. For fermented grain, 5 g of fermented grain is weighed, 20 mL ultrapure water is added in the fermented grain to obtain a mixture, the mixture is shaken to mix thoroughly, and then ultrasonicated in an ice bath for 30 minutes to obtain an ultrasonicated mixture, the ultrasonicated mixture is centrifuged at 4° C. and 8000×g for 5 minutes, a supernatant is taken and filtered with a 0.22 μm aqueous-phase membrane to obtain a filtrate, and the filtrate is transferred to an HPLC vial. HPLC system: Waters 2695; chromatographic column: Bio-Rad Aminex HPX-87H Ion Exclusion Column; column temperature: 60° C.; detector: photodiode array (PDA) detector, detection wavelength: 210 nm; mobile phase: 5 mmol L⁻¹ H₂SO₄; flow rate: 0.6 mL/minute.

### (III) Method for Ethanol Content Determination

For a fermentation broth obtained from a simulation experiments, the fermentation broth is filtered with a 0.22 μm aqueous-phase membrane filter to obtain a filtrate, and the filtrate is transferred to a high-performance liquid chromatography (HPLC) vial. For fermented grain, 5 g of fermented grain is weighed, 20 mL ultrapure water is added in the fermented grain to obtain a mixture, the mixture is shaken to mix thoroughly, and then ultrasonicated in an ice bath for 30 minutes to obtain an ultrasonicated mixture, the ultrasonicated mixture is centrifuged at 4° C. and 8000×g for 5 minutes, a supernatant is taken and filtered with a 0.22 μm aqueous-phase membrane to obtain a filtrate, and the filtrate is transferred to an HPLC vial. HPLC system: Waters 2695; chromatographic column: Bio-Rad Aminex HPX-87H Ion Exclusion Column; column temperature: 60° C.; detector: differential refractive index detector (RID), mobile phase: 5 mmol L⁻¹ H₂SO₄; flow rate: 0.6 mL/minute.

### (IV) Method for Volatile Compound Determination

For a fermentation broth obtained from a simulation experiments, the fermentation broth is centrifuged at 8000×g and 4° C. for 5 minutes, 8 mL of a supernatant is taken and transferred to a headspace vial. 5 g of fermented grain is weighed, 20 mL ultrapure water is added in the fermented grain, 0.85% NaCl and 1% CaCl₂) are added to obtain a mixture, the mixture is shaken to mix thoroughly, and then ultrasonicated in an ice bath for 30 minutes to obtain an ultrasonicated mixture, the ultrasonicated mixture is centrifuged at 4° C. and 8000×g for 5 minutes, 8 mL of a supernatant is taken and transferred in a headspace vial (20 mL, 3 g NaCl is added in advance), and then 20 μL L-menthol (100 μg/mL) is added. Volatile compounds are detected by HS-SPME-GC-MS (GC 6890N and MS 5975) and DB-Wax column (30 m×0.25 mm, 0.25 μm).

### Example 1: Strain Screening

A strain of lactic-acid-tolerant Zygosaccharomyces bailii was screened from a fermentation process of Maotai-flavor liquor, with the screening process as follows:

A sample of fermented grain from a well-known Maotai-flavor liquor was collected, 10 g of the sample was taken and added in 90 mL of 0.9% sterile saline to obtain a mixture, the mixture was shaken at 30° C. and 200 rpm for 1 hour, a suspension was taken and subjected to serial 10-fold dilutions; and 100 μL of the diluted liquid was evenly spread onto a YPD solid plate; after culturing for 3 days, single colonies were picked for purification. The resulting single colonies were added to sorghum juice medium with pH 3.2 adjusted by lactic acid, and cultured at 30° C. and 200 rpm for 3 days. After the culture was completed, growth amount and lactic acid, ethanol, and volatile compounds in the fermentation broth were measured.

Finally, a strain capable of degradation of lactic acid and achieving high-yield production of ethanol and flavor compounds was selected. The strain was streaked onto YPD slant medium for preservation and also stored in glycerol stock.

### Example 2: Strain Identification

### (1) Colony Characteristics and Microbial Morphology

The strain obtained in Example 1 was activated and streaked onto YPD solid medium, and then incubated statically at 30° C. for 2 days. Colony morphology of the strain on the plate was observed. At the same time, a single colony was also picked and placed in sorghum juice liquid medium for culturing, and observation under transmission electron microscopy was performed when the colony was cultured for 2 days.

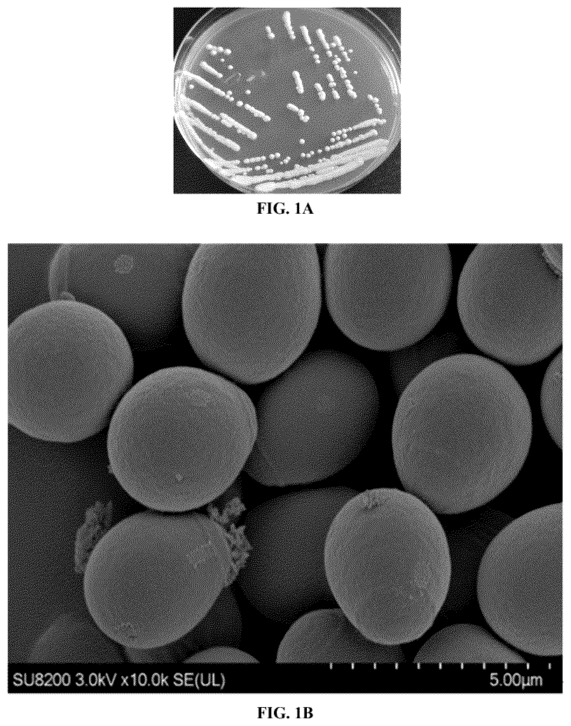

As shown in FIG. 1, the colony morphology of the strain on the YPD plate is white and opaque with a certain gloss; and the colony has regular, smooth, and circular edges with a bulge in a center. Under the transmission electron microscopy, a single cell is relatively large and oval-shaped, without flagella or mycelia.

### (2) Growth Characteristics

The strain was inoculated into sorghum juice medium and cultured at 30° C., 200 rpm for 84 hours, and samples were taken every 12 hours to measure OD₆₀₀ to monitor its growth characteristics.

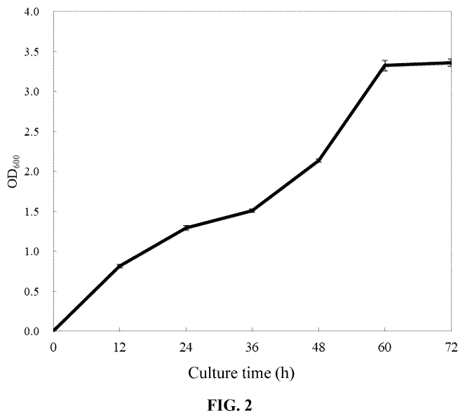

As shown in FIG. 2, the strain reached a stationary phase after 60 hours of growth in the sorghum juice medium, with an OD₆₀₀ of 3.36±0.05 at the end of fermentation. A glucose content in the sorghum juice medium at the beginning of fermentation was 40 g/L. Results in Table 1 show that residual glucose at the end of fermentation was 4.09±0.23 g/L, and the ethanol production of the strain was 13.66±0.45 g/L.

TABLE 1
Glucose and ethanol content in fermentation
broth at the end of fermentation (n = 3)

| Substance | Content (g/L) |
|---|---|
| Glucose | 4.09 ± 0.23 |
| Ethanol | 13.66 ± 0.45 |

### (3) Physiological and Biochemical Experiments and Carbon Source Utilization Experiments.

The carbon source utilization of the strain was determined as follows: a single colony was picked and inoculated into sorghum juice medium and cultured at 30° C., 200 rpm for 24 hours to prepare and obtain a seed culture. 10 mL of the seed culture was taken and centrifuged at 10,000×g for 5 minutes, a supernatant was discarded, a cell pellet was suspended in an equal volume of sterile saline to obtain a suspension, the suspension was then diluted to 10⁻⁴ with the same saline, and 100 μL of the diluted suspension was taken and inoculated into a Biolog YT microplate. According to the instructions, the microplate was incubated statically in a 30° C. constant temperature incubator for 36 hours, and an OD₅₉₀ was measured using a microplate reader. An OD₅₉₀>0.2 indicates that the strain can utilize the carbon source.

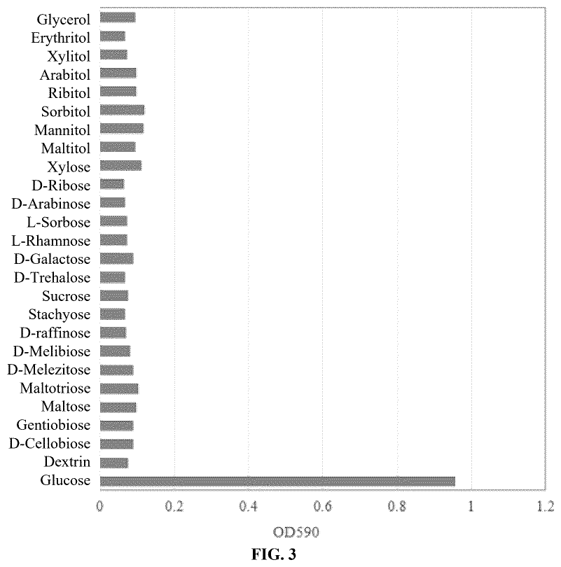

As shown in FIG. 3, the strain has a relatively narrow carbon source utilization spectrum, utilizing only glucose among the measured carbon sources, with an OD₅₉₀ value of 0.96, For other carbon sources, the values are all lower than 0.2.

### (4) Molecular Biology Identification

A single colony of the strain was inoculated into YPD liquid medium for culture, and total DNA was then extracted after the colony was cultured for 48 hours. PCR amplification was performed using ITS universal primers (ITS1 and ITS4). PCR amplification conditions were as follows: 30 cycles at 94° C. for 5 minutes, 94° C. for 30 seconds, 55° C. for 30 seconds, and 72° C. for 1 minute; and then at 72° C. for 10 minutes. The PCR amplification products were verified by electrophoresis on 1% agarose gel and then sent to Suzhou Genewiz Biotechnology Co., Ltd. for sequencing. The sequencing results were submitted to the National Center for Biotechnology Information (NCBI) database for BLAST analysis. The strain exhibited a highest homology with Zygosaccharomyces bailii, therefore, the strain was identified as Zygosaccharomyces bailii, designated as Zygosaccharomyces bailii LBM888.

The strain was deposited with China Center for Type Culture Collection on May 10, 2023, with a deposit address of Wuhan University, Wuhan City, P. R. China, and an accession number of CCTCC NO: M 2023729.

### Example 3: Ethanol and Flavor Compound Production Capacity of Strain CCTCC NO: M 2023729 Under Lactic Acid Stress

A pH value of sorghum juice medium was adjusted to 3.2 with lactic acid to serve as a fermentation medium for evaluating the ethanol and flavor compound production capacity of the experimental strain Zygosaccharomyces bailii CCTCC NO: M 2023729 (hereinafter referred to as Zygosaccharomyces bailii ZB) under lactic acid stress.

Specific steps were as follows:

A single colony of the Zygosaccharomyces bailii CCTCC NO: M 2023729 was inoculated into sorghum juice medium and cultured at 30° C. and 200 rpm for 24 hours to obtain a seed culture.

1 mL of the seed culture of Zygosaccharomyces bailii ZB was inoculated into 100 mL of a fermentation medium, which was added with 40 g/L lactic acid (a pH value of the sorghum juice medium was adjusted to pH 3.2 with lactic acid), and cultured at 30° C. and 200 rpm for 72 hours, samples were taken every 12 hours to measure the growth (OD₆₀₀) of strain, lactic acid, ethanol, and volatile flavor compounds in the fermentation broth.

In addition, a fermentation medium without lactic acid was used as a control.

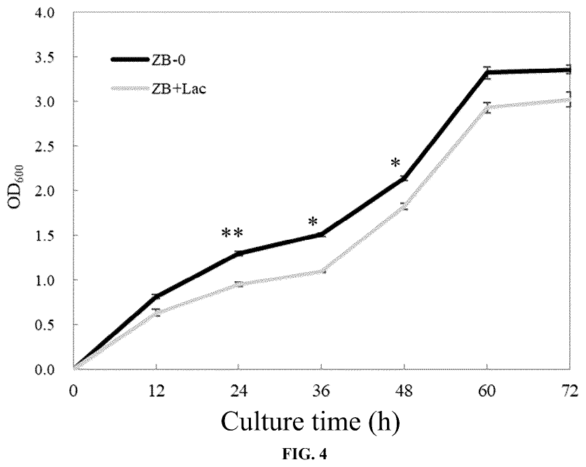

Results showed that the strain Zygosaccharomyces bailii ZB could adapt to lactic acid stress and maintain robust growth under lactic acid stress, as shown in FIG. 4. Although the growth of Zygosaccharomyces bailii ZB under lactic acid stress was significantly lower than (P<0.05) that without lactic acid stress in the first 48 hours of fermentation, but no significant difference in biomass was observed at the end of fermentation (P>0.05), with OD₆₀₀ values of 3.02±0.08 and 3.36±0.05 for with lactic acid stress and without lactic acid stress, respectively.

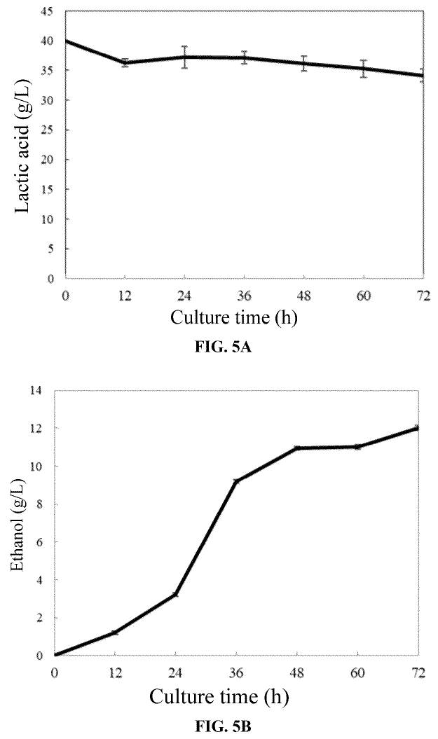

Determination of lactic acid content in the fermentation broth indicated that a primary mechanism for tolerance of Zygosaccharomyces bailii ZB to lactic acid was its ability to degrade lactic acid present in the system (FIG. 5A). The initial lactic acid content at the beginning of fermentation was 40 g/L, which decreased to 34.12±1.07 g/L at the end of fermentation, with a degradation of 5.88 g/L. Results of ethanol determination showed that Zygosaccharomyces bailii ZB could not only adapt to 40 g/L lactic acid stress but also produced a high content of ethanol under lactic acid stress, and the content of ethanol reached 11.99±0.13 g/L at the end of fermentation (FIG. 5B).

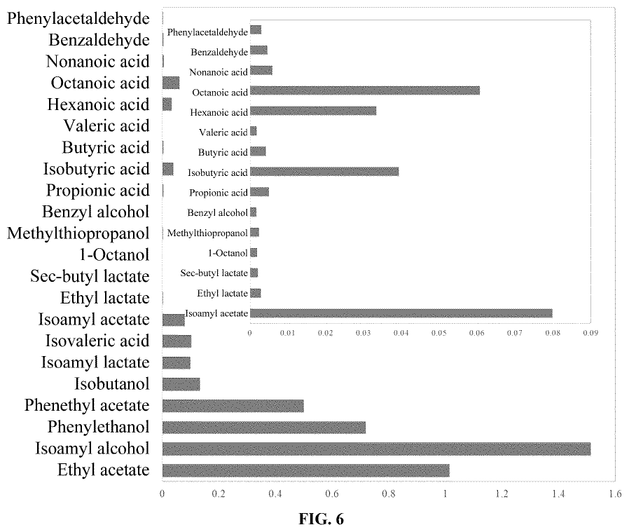

As shown in FIG. 6, the results indicate that Zygosaccharomyces bailii CCTCC NO: M 2023729 could produce a variety of flavor compounds under lactic acid stress. Flavor compounds with a content exceeding 0.1 g/L included ethyl acetate (1.02 g/L), isoamyl alcohol (1.52 g/L), phenylethyl alcohol (0.72 g/L), phenylethyl acetate (0.50 g/L), isobutanol (0.13 g/L), isoamyl lactate (0.10 g/L), and isovaleric acid (0.10 g/L). Flavor compounds with a content below 0.1 g/L included isoamyl acetate, ethyl lactate, and 1-octanol.

The above results demonstrate that Zygosaccharomyces bailii CCTCC NO: M 2023729 could adapt well to a lactic acid environment, with the ability to reduce lactic acid content in the system while producing ethanol and various flavor compounds.

### Example 4: Application of Yeast Agent in Baijiu (Chinese Liquor) Brewing

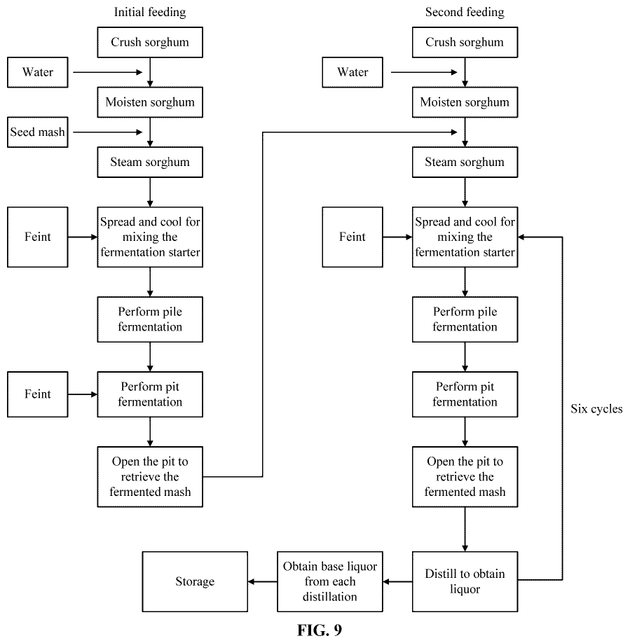

From initial feeding of sorghum to a last round of liquor extraction and dreg disposal, the production of Maotai-flavor liquor follows a law of seasonal succession. One production cycle lasts for nearly one year, which involves two cycles of feeding, that is, initial feeding of sorghum and second feeding of sorghum, nine times of boiling and steaming, eight times of fermentation, and seven times of liquor extraction, and the production process is shown in FIG. 9.

The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension. The yeast suspension was applied to a fourth fermentation round of the Maotai-flavor liquor process.

The specific operating steps are as follows:

After the fermented grain was distilled, spread and mixed with Qu (Chinese fermentation starter), 30 kg of the fermented grain was taken out, and 1000 mL of the yeast suspension was evenly sprayed onto the 30 kg fermented grain and thoroughly mixed. 1000 mL of sterile water was evenly spread onto 30 kg of the fermented grain to obtain a blank control.

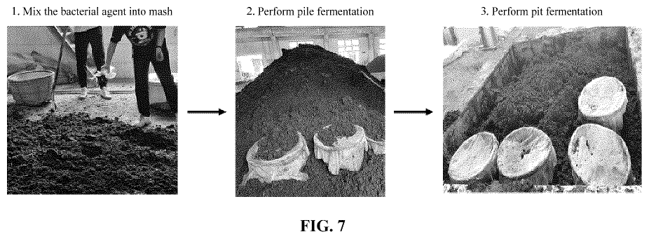

Each treatment was performed in three replicates, the prepared fermented grain was finally placed into a bamboo basket and placed in a fermentation pile and a fermentation pit for in-situ fermentation, as shown in FIG. 7.

Samples were collected at the end of pile fermentation and at the end of pit fermentation to determine lactic acid, ethanol, and volatile flavor compounds in the fermented grain.

Results are as follows:

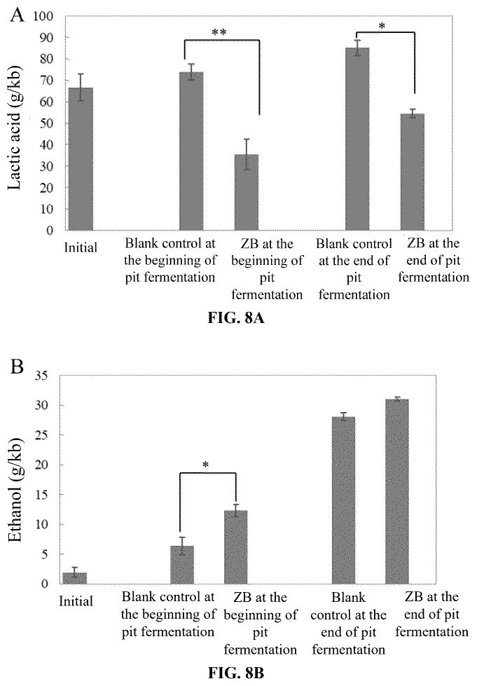

Lactic acid and ethanol determination results are shown in FIG. 8. It can be seen that the initial lactic acid content of a fourth round of fermentation of the Maotai-flavor liquor was higher than 60 g/kg, indicating that lactic acid accumulation is an important factor restricting the yield and quality of Maotai-flavor liquor.

Addition of the experimental yeast suspension effectively reduced lactic acid content during fermentation (FIG. 8A). At the end of pile fermentation, lactic acid content of the group without the yeast suspension (blank control at the beginning of pit fermentation) was 73.84±3.65 g/kg, while lactic acid content of the group with yeast suspension (ZB at the beginning of pit fermentation) was 35.45±7.09 g/kg, a decrease of 51.99%.

At the end of pit fermentation, lactic acid content of the control group (blank control at the end of pit fermentation) was 85.07±3.52 g/kg, while lactic acid content of the group with yeast suspension (ZB at the end of pit fermentation) was 54.51±2.08 g/kg, a decrease of 35.92%.

For ethanol production, the addition of yeast suspension significantly (P<0.05) increased ethanol content during file fermentation (FIG. 8B). At the end of pile fermentation completion, ethanol content of the control group was 6.37±1.45 g/kg, and ethanol content of the group added with yeast suspension was 12.30±1.01 g/kg, an increase of 92.93%. At the end of pit fermentation, ethanol content of the control group was 28.00±0.65 g/kg, while ethanol content of the group with yeast suspension was 29.98±0.34 g/kg, an increase of 7.07%.

In addition, addition of the yeast suspension significantly increased volatile flavor compounds in the fermented grain, mainly including esters, alcohols, and aromatic compounds, as shown in Table 2. Significantly increased esters included ethyl acetate, ethyl propionate, isoamyl acetate, ethyl isobutyrate, furfuryl acetate, ethyl phenylacetate and phenethyl acetate, and the like. Significantly increased alcohols included isobutyl alcohol, phenylethyl alcohol, isoamyl alcohol, and 2-heptanol. Significantly increased aromatic compounds included 2-ethyl-6-methylpyrazine, 2,6-diethylpyrazine, and tetramethylpyrazine.

TABLE 2
Effects of addition of CCTCC NO: M 2023729 on volatile flavor
compounds during the fermentation of Maotai-flavor liquor

| | | Blank | | Blank | |
|---|---|---|---|---|---|
| | | control at | ZB at | control at | ZB at |
| | | the beginning | the beginning | the end | the end |
| | | of pit | of pit | of pit | of pit |
| Flavor compounds | Initial | fermentation | fermentation | fermentation | fermentation |
| Esters (mg/kg) | | | | | |
| Ethyl acetate | 2.18 ± 0.04 | 3.78 ± 0.06 | 4.74 ± 0.35 | 9.36 ± 2.47 | 11.24 ± 1.18 |
| Ethyl propionate | 0.02 ± 0.00 | 0.11 ± 0.00 | 0.61 ± 0.05 | 0.19 ± 0.02 | 0.88 ± 0.05 |
| Ethyl | 0.00 ± 0.00 | 0.00 ± 0.00 | 0.00 ± 0.00 | 0.00 ± 0.00 | 0.22 ± 0.01 |
| 2-methylpropanoate | | | | | |
| Isoamyl acetate | 0.09 ± 0.02 | 0.21 ± 0.04 | 0.30 ± 0.04 | 0.55 ± 0.22 | 0.72 ± 0.23 |
| Furfuryl acetate | 0.00 ± 0.00 | 0.06 ± 0.01 | 0.20 ± 0.00 | 0.10 ± 0.00 | 0.45 ± 0.02 |
| 2-methoxyacetic acid | 0.03 ± 0.00 | 0.19 ± 0.00 | 0.21 ± 0.00 | 0.56 ± 0.04 | 1.21 ± 0.09 |
| 3-methylbutyl ester | | | | | |
| Ethyl phenylacetate | 0.14 ± 0.02 | 1.67 ± 0.10 | 1.92 ± 0.05 | 6.08 ± 0.09 | 7.22 ± 0.30 |
| Diethyl glutarate | 0.00 ± 0.00 | 0.00 ± 0.00 | 0.00 ± 0.00 | 0.09 ± 0.00 | 0.14 ± 0.00 |
| Butyl lactate | 0.00 ± 0.00 | 0.00 ± 0.00 | 0.03 ± 0.00 | 0.00 ± 0.00 | 0.12 ± 0.12 |
| Ethyl | 0.00 ± 0.00 | 0.05 ± 0.00 | 0.12 ± 0.00 | 0.11 ± 0.00 | 0.55 ± 0.00 |
| 3-phenylpropionate | | | | | |
| Ethyl palmitate | 0.40 ± 0.03 | 1.81 ± 0.09 | 2.83 ± 0.10 | 5.25 ± 0.21 | 6.55 ± 0.22 |
| Ethyl hexadecenoate | 0.00 ± 0.00 | 0.08 ± 0.00 | 0.10 ± 0.00 | 0.15 ± 0.01 | 0.26 ± 0.00 |
| Phenethyl acetate | 0.09 ± 0.00 | 0.63 ± 0.02 | 1.33 ± 0.10 | 0.91 ± 0.07 | 2.44 ± 0.11 |
| Ethyl linoleate | 0.20 ± 0.05 | 0.43 ± 0.02 | 0.74 ± 0.06 | 1.19 ± 0.09 | 1.54 ± 0.05 |
| Alcohols (mg/kg) | | | | | |
| Isobutyl alcohol | 0.00 ± 0.00 | 0.05 ± 0.00 | 0.30 ± 0.01 | 0.10 ± 0.01 | 0.31 ± 0.03 |
| Phenylethyl alcohol | 2.98 ± 0.09 | 5.53 ± 2.73 | 11.06 ± 1.40 | 7.97 ± 2.45 | 29.23 ± 2.87 |
| Isoamyl alcohol | 0.98 ± 0.04 | 1.09 ± 0.32 | 3.82 ± 0.47 | 1.98 ± 0.58 | 4.60 ± 1.09 |
| 2-heptanol | 0.00 ± 0.00 | 0.00 ± 0.00 | 0.10 ± 0.01 | 0.00 ± 0.00 | 0.13 ± 0.01 |
| Aromatic compounds | | | | | |
| (mg/kg) | | | | | |
| 2-ethyl-6-methylpyrazine | 0.00 ± 0.00 | 0.00 ± 0.00 | 0.06 ± 0.01 | 0.04 ± 0.01 | 0.19 ± 0.00 |
| 2,6-diethylpyrazine | 0.00 ± 0.00 | 0.00 ± 0.00 | 0.04 ± 0.00 | 0.09 ± 0.02 | 0.20 ± 0.01 |
| Tetramethylpyrazine | 0.07 ± 0.00 | 0.17 ± 0.09 | 0.51 ± 0.06 | 1.01 ± 0.08 | 1.51 ± 0.02 |

### Example 5: Application of Yeast Agent in Baijiu (Chinese Liquor) Brewing

Specific steps were as follows:

The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

### (1) Daqu Light-Flavor Baijiu:

The prepared yeast suspension was mixed with grain that had been soaked, steamed and cooled, followed by thorough mixing with starter for fermentation. A dosage of the yeast suspension was 10-20% of the total feeding amount (by mass), and the mixture was then transferred into a fermentation tank for fermentation. Raw and fermented materials were distilled separately and then fermented separately to prepare Daqu light-flavor Baijiu (For the specific production process of Daqu, please refer to Research Progress on Microorganisms and Flavor Compounds in Light-flavor Baijiu Brewing).

### (2) Xiaoqu Light-Flavor Baijiu:

The prepared yeast suspension was mixed with grain that had been steamed and cooled, and then then subjected to saccharification by piling for 1 day, and the mixture was thoroughly blended with starter and transferred into a fermentation tank for 14 days of fermentation. A dosage of the solid-state bacteria was 1% of the total feeding amount (by mass). Raw and fermented materials were distilled apart and then fermented apart to prepare Xiaoqu light-flavor Baijiu (For the specific production process of Xiaoqu, please refer to Metabolic Characteristics of Microbial Flora Synthesizing Higher Alcohols during Fermentation Process of Xiaoqu Light-flavor Baijiu).

### (3) Laobaigan-Flavor Baijiu

The grain that had been steamed and cooled was blended, and the fermented grain was mixed and re-steamed. The prepared yeast suspension was added to the fermented grain, an addition amount of the yeast suspension was 25% of the total feeding amount (by mass), and the mixture was then transferred into a fermentation tank for 40 days of fermentation. Raw and fermented materials were distilled apart and fermented together (For the specific production process of Laobaigan-flavor Baijiu, please refer to Optimization of Formula for Multi-Grain Formulation of Laobaigan-flavor Baijiu).

Results indicate that the yeast suspension of the present invention can increases the flavor compounds in Baijiu.

### Example 6: Application of Yeast Agent in Huangjiu (Cooking Wine) Brewing

(1) The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

(2) Preparation of distiller's yeast: A saccharified liquid was prepared first, glutinous rice was soaked in a rice soaking tank for 30 minutes, rice-soaked water was discarded, the soaked rice was steamed for 30-40 minutes, and the steamed rice was cooled to 60° C. and set aside for later use; and 2% % saccharification enzyme and 1% % liquefaction enzyme were added into the cooled rice and mixed thoroughly, the mixture was then placed in a water bath at 60° C. for saccharification at a constant temperature for more than 4 hours, and the mixture was stirred every 1 hour to ensure uniform saccharification. The saccharified liquid was filtered through cheesecloth to obtain a saccharified filtrate, and the saccharified filtrate was diluted with water to 13±0.5° Bx (20° C.). The diluted mixture was poured into a bottle, which was sealed with film, and placed into a sterilizer to sterilize at 121° C. for 20 minutes, and the bottle was then removed and cooled for later use. The cultured yeast suspension was inoculated into the rice saccharified liquid at an inoculation ratio of 5% (v/v) and cultured at a constant temperature for 36-48 hours before being used for grain feeding.

(3) Fermentation of Huangjiu: Ingredients were added according to the Huangjiu brewing formulation: 1000 g glutinous rice, 1500 g cooked rice, 1100 mL brewing water, 110 g Daqu starter, and 100 mL distiller's yeast.

The fermentation lasted for about 20 days in total. During fermentation, raking (opening the fermented grain) was performed, a temperature of main fermentation was controlled below 33° C., and a temperature of secondary fermentation was controlled at 15±2° C. A temperature of main fermentation was 28° C., and the main fermentation lasted for a total of 5 days with raking twice daily; and a temperature of secondary fermentation was 15° C., and the secondary fermentation lasted for 15 days with raking once every 5 days.

Results indicate that the yeast suspension of the present invention can increases the flavor compounds in Huangjiu (cooking wine).

### Example 7: Application of Yeast Agent in Soy Sauce Brewing

(1) The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

(2) Plump and clean soybeans were selected, washed and soaked in water for 5-10 hours; after the soybeans were fully hydrated and swollen, the soaked soybeans were drained out and steamed under pressure until cooked; the hot soybeans were taken out and cooled to about 70-80° C., 30-40% flour was added (by mass of the soybeans) and stirred thoroughly, and continued cooling to 30-40° C.; 2%-5% (v/v) of the yeast suspension was then added, and mixed thoroughly to obtain a mixture, and the mixture was fermented at about 28-30° C. During fermentation, a fermentation starter needed to be continuously mixed and ventilated to dissipate heat generated by microbial metabolism. After 72-96 hours, a soy sauce fermentation starter was obtained. Salt water was added to the prepared soy sauce fermentation starter at a ratio of 1:1.5 (m/v), and stirred thoroughly for fermented grain preparation, and fermented for 3-6 months, and the fermented grain was stirred every 15 days. Upon completion of fermentation, the soy sauce fermented grain was pressed, filtered to remove solids and impurities, and sterilized to obtain the finished soy sauce.

Results indicate that the yeast suspension of the present invention can increases the flavor compounds in the soy sauce.

### Example 8: Application of Yeast Agent in Vinegar Brewing

(1) The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

(2) Material selection and processing: 100 kg of high-quality glutinous rice was selected and soaked with 200 kg of water for 15-24 hours, the soaked rice was rinsed until the rinse water was clear, and the rice was drained and steamed, and then water was sprayed on the cooked rice to cool to 25-30° C.;

Mixing and transferring into a fermentation tank: before the cooled rice was transferred into the fermentation tank, 1.5-2 kg of fermentation starter was added in the cooled rice, which was saccharified at a low temperature for 72-96 hours;

Alcohol fermentation: after saccharification, 30 kg of water and 6 kg of the yeast suspension were added, and fermented at 28° C. for 144-168 hours to obtain mature alcoholic fermented grain;

Preparation of vinegar mash: 150 kg of wheat bran was added in a fermentation pool and flattened, the mature alcoholic mash was poured into the fermentation pool and stirred thoroughly, 5 kg of rice husks was evenly spread on an upper layer of the pool, 5 kg of the mature alcoholic fermented grain from the previous batch was taken, added and stirred thoroughly to cover the 5 kg rice husks, and spread evenly to complete the reparation of vinegar mash;

Acetic acid fermentation: the vinegar mash was turned every 24 hours of fermentation, rice husks were added to keep warm and moisture after each turning; rice husks were not added any more from the 11^{th} day of fermentation, the vinegar mash was turned to lower down a pile temperature; and after 18-20 days, when acidity no longer increased, 4 kg of salt was added and sealed for 30-45 days;

Vinegar leaching and boiling: the vinegar mash after ageing was taken, toasted rice colorant was added in proportion, and soaked for several hours; the vinegar mash was leached using a countercurrent leaching method to extract vinegar juice; and sugar was then added to the extracted vinegar juice for flavor adjustment, followed by clarification and boiling, and the vinegar was bottled and sealed when a pile temperature cooled down to 75-80° C.

Results indicate that the yeast suspension of the present invention can increases the flavor compounds in the vinegar.

### Example 9: Application of Yeast Agent in Bright Huangjiu Brewing

The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

(1) Polishing: 20-30% of the raw rice weight was removed using a polishing machine. Broken rice grains were minimized; polished rice and brown rice should have similar shape, and germ and groove of the raw rice should be completely removed;

(2) Soaking: the polished rice was soaked in water with room temperature until water absorption reached 28-30%, and soaking time was 9-12 hours;

(3) Steaming: the soaked rice was steamed to obtain grains that were distinct, firm on the outside and soft inside, with no white core; the rice should be loose but not mushy, cooked but not overdone, and the steamed glutinous rice yield was generally 140-150%;

(4) Ingredient loading: the steamed rice was cooked to 25-30° C. when ingredient loading was performed, including 100 kg of raw rice, 140 kg of steamed rice, rice fermentation starter, 20 kg of yeast suspension, 3 kg of distiller's yeast starter, and 120 L of water; and an adding amount of rice fermentation starter was 20%-30% of a weight of the raw rice (that is, polished white rice).

(5) Fermentation: an initial fermentation temperature was controlled at 28-30° C. for 3 days, then fermented at about 20° C. for 22 days, with total fermentation time of 25 days.

Results indicate that the yeast suspension of the present invention can increases the flavor compounds in the bright Huangjiu.

### Example 10: Application of Yeast Agent in Baijiu Brewing

The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

(1) Sorghum (40-50%), wheat (6-10%), corn (5-10%), glutinous rice (10-12%), and rice (15-20%) were weighed according to specified proportions, mixed together, and soaked in hot water (55-65° C.) for 24-32 hours; and rice husks (20-35% of the total grain weight) were then added and steamed at 90-100° C. for 30-40 minutes and cooled for later use;

(2) Primary steaming: The soaked corn, sorghum, and wheat were drained to remove surface moisture for primary steaming, with steaming time for 30-60 minutes;

(3) Stewing: After primary steaming, warm water at 40-50° C. was added to cover the grain with a water level at least 15 cm above a grain surface, the grains were simmered for 10-30 minutes, and the water was then drained;

(4) Secondary steaming: The primary-steamed grains were mixed thoroughly with the rice and glutinous rice that had been soaked and moistened in the step (1), and then subjected to secondary steaming for 60-70 minutes;

(5) Cooling and addition of fermentation starter: The steamed mash was cooled to 30-35° C., the treated rice husks obtained in the step (1) and 18-28% Baijiu Daqu were added, and 5-10% yeast suspension (calculated according to the weight of grains) was added to obtain a mixture, and the mixture was stirred evenly and transferred to a fermentation pit for fermentation;

(6) Pit sealing and fermentation: After of transferred into the fermentation pit, the mash was covered with a grain layer with at least 40 cm above a ground level, and then sealed with pit mud with a thickness of 15-20 cm, and the fermentation lasted for 45-70 days; and

(7) Distillation: The fermented grain was removed from the fermentation pit and transferred into a distillation pot for distillation to extract liquor, a distillation flow rate was 1.5-2.5 kg/minute, and a liquor distillate temperature was 25-30° C.

Baijiu produced according to the above process had a liquor yield of 28-33% and a high-quality product rate of 88-91%. The resulting Baijiu was colorless or slightly yellow and clear, with prominent ester and caramel aromas. It had a clean and refreshing taste, a sweet and mellow mouthfeel, and a rich, multi-grain puffed aroma that was naturally integrated. The liquor had a mellow body characterized by the typical style of “Ru” (mellow), “Ya” (elegant), and “Rong” (harmonious). The resulting Taorong-type Baijiu met the first-class liquor quality standard.

### Example 11: Application of Yeast Agent in Sweet Fermented Rice Brewing

The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

Rice was weighed as a main raw material according to a specified proportion and soaked at room temperature for 8-12 hours until the rice fully absorbed water; the soaked rice was drained, then steamed under atmospheric pressure for 30-40 minutes until no white core remained in the rice grains; after steaming, the rice was rinsed with cooled boiled water until a temperature reached 25-28° C., and then transferred to a fermentation tank; 0.4%-0.8% (mass ratio) of the yeast suspension was added in proportion into the fermentation tank, 0.5%-1.5% (mass ratio) of the distiller's yeast was inoculated, the mixture was stirred evenly, and drinking water at 1-1.5 times the weight of raw rice was added; and fermentation was carried out at a constant temperature of 28-30° C. for 48-72 hours. The resulting sweet fermented rice had an alcohol content of 2%-4% (v/v), with harmonious and rich aroma, uniform texture, and a pleasantly sweet and sour taste.

Results indicate that the yeast suspension of the present invention can increases the flavor compounds in the sweet fermented rice.

### Example 12: Application of Yeast Agent in Rice Wine

The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

Rice was weighed as a main raw material according to a specified proportion and soaked at room temperature for 8-12 hours until the rice fully absorbed water; the soaked rice should be easily crushed by hand without hard core remaining; the soaked rice was drained, then steamed under atmospheric pressure for 30-40 minutes until no white core remained in the rice grains; after steaming, the rice was rinsed with cooled boiled water until a temperature reached 25-28° C., and 5%-10% (mass ratio) of the yeast suspension was added in proportion for saccharification; after saccharification, 5%-10% of the distiller's yeast was inoculated, the mixture was stirred evenly, and drinking water at 1-1.5 times the weight of raw rice was added; and fermentation was carried out at a temperature of 28-30° C. for 3-5 days, followed by fermentation at a temperature of 10-15° C. for 10-15 days; and after fermentation, the mash was pressed and filtered to obtain rice wine. The resulting rice wine had an alcohol content of 5%-10% (v/v), was rich in rice and fruit aroma, harmonious and rich aroma, and uniform texture.

Results indicate that the yeast suspension of the present invention can increases the flavor compounds in rice wine.

### Example 13: Application of Yeast Agent in Fermented Dairy Products

The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

It can be used to manufacture lactobacillus fermented milk beverages. The specific preparation process was as follows:

Skimmed milk, used as raw material, was sterilized at 95° C. for 20 minutes, then cooled to 4° C., and the yeast suspension was then added to make a viable cell concentration reach at least 10⁶ CFU/mL or more, and stored at 4° C. to obtain the fermented milk beverage.

Results indicate that the yeast suspension of the present invention can increases the flavor compounds in the milk beverage.

### Example 14: Application of Yeast Agent in Fermented Dairy Products

The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

It can be used to manufacture soy milk. The specific preparation process was as follows:

Soybeans were soaked in soft water, with a water volume in three times a volume of original soybeans, and the soybeans were soaked at a temperature of 80° C. for 1-2 hours. Soybean skins were then removed. The soaked water was drained, and boiled water was added to grind the soybeans into slurry, which was maintained above 80° C. for 10-15 minutes. The slurry was filtered through a 150-mesh filter membrane and centrifuged to obtain crude soy milk, the crude soy milk was heated to 140-150° C., and the crude soy milk was rapidly transferred into a vacuum cooling chamber for vacuum treatment. Odorous substances in the crude soy milk were quickly discharged along with water vapor. After vacuum degassing, the temperature was lowered to about 37° C., and the yeast suspension was added to achieve a concentration of at least 10⁶ CFU/mL, and the product was stored at 4° C. to obtain the soy milk.

Results indicate that the yeast suspension of the present invention can increases the flavor compounds in soy milk.

### Example 15: Application of Yeast Agent in Fermented Fruit and Vegetable Beverages

The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

It can be used to manufacture fermented fruit and vegetable beverages. The specific preparation process was as follows:

Fresh vegetables (such as one or more of cucumber, carrot, beetroot, celery, or cabbage) were washed and juiced, the juice was then subjected to high-temperature instantaneous sterilization by heating at 140° C. for 2 seconds, followed immediately by cooling to about 37° C. The yeast suspension of the present invention was added to achieve a viable cell concentration of at least 10⁶ CFU/mL, and the product was stored at 4° C. to obtain the fermented fruit and vegetable beverage.

Results indicate that the yeast suspension of the present invention can increases the flavor compounds in fermented fruit and vegetable beverages.

### Example 16: Application of Yeast Agent in Whiskey Brewing

Main steps were as follows:

The Zygosaccharomyces bailii CCTCC NO: M 2023729 (hereinafter referred to as Zygosaccharomyces bailii ZB) was inoculated into seed culture medium and cultured at 30° C. and 200 rpm for 48 hours to obtain a seed culture of 1×10⁸ cells/mL. 10 mL of the seed culture was taken and centrifuged at 10,000×g for 5 minutes, a supernatant was discarded to obtain a precipitate, and the precipitate was resuspended in 1000 mL sterile water to prepare a yeast suspension.

Milling: The weighed malt was mechanically milled and processed into coarse granules;

Saccharification: The milled malt was mixed with hot water at a temperature of 63.5° C., and then saccharified to obtain malt wort.

Fermentation: After cooling, the malt wort was pumped into a fermentation tank, 20% (v/v) of the yeast suspension was added for fermentation, and fermentation time is 56-60 hours.

Distillation: The fermented malt wort was distilled twice in a copper Holstein still. First distillation: The fermented liquid was added into a distillation kettle; when a temperature inside the kettle reached 80° C., the fermented liquid started to boiling, the distillation stage began; a temperature of cooling water was maintained below 18° C.; neither foreshots nor feints were separated in the first distillation; a distillate alcohol concentration was 20%-28% vol, and time of the first distillation was controlled at 8-9 hours. Second distillation: an initial distillate was distilled again to separate foreshots, hearts, and feints; the hearts were retained and reduced to 63.5% vol Component A, which was reserved for blending a final product; and the foreshots and feints were used for a next distillation. Time of the second distillation was about 10-12 hours.

Aging: Aging was performed after the distillation.

Blending and filtration: The alcohol content was adjusted to 40 degrees to obtain a semi-finished product, which was filtered to obtain the final whiskey product.

Results indicate that the yeast suspension of the present invention can increases the flavor compounds in the whiskey.

Although the present invention has been disclosed as above in the form of preferred embodiments, it is not intended to limit the present invention. Those skilled in the art can make various modifications and variations without departing from the spirit and scope of the present invention. Therefore, the scope of protection of the present invention should be defined by the claims.

What is claimed is:

1. A microbial agent, comprising Zygosaccharomyces bailii, wherein the Zygosaccharomyces bailii was deposited with China Center for Type Culture Collection (CCTCC) on May 10, 2023 at Wuhan University, Wuhan City, P. R. China, with an accession number of CCTCC NO: M 2023729.

2. The microbial agent according to claim 1, wherein the microbial agent uses the Zygosaccharomyces bailii CCTCC NO: M 2023729 as a principal microorganism.

3. The microbial agent according to claim 2, wherein a viable count of the Zygosaccharomyces bailii CCTCC NO: M 2023729 in the microbial agent is 10⁵-10⁸ CFU/mL.

4. The microbial agent according to claim 3, wherein a viable cell concentration of the Zygosaccharomyces bailii CCTCC NO: M 2023729 in the microbial agent has a range of 10⁵-10⁸ CFU/mL.

5. The microbial agent according to claim 1, wherein the microbial agent is a liquid microbial agent or a solid microbial agent.

6. The microbial agent according to claim 1, comprising viable cells, dried cells obtained by freeze drying, and immobilized cells of the Zygosaccharomyces bailii CCTCC NO: M 2023729 strain.

7. Application of Zygosaccharomyces bailii CCTCC NO: M 2023729 or a microbial agent containing the Zygosaccharomyces bailii CCTCC NO: M 2023729, wherein the application is one or more of the following: degradation of lactic acid, increase in an ethanol content, and increase in contents of flavor compounds, and the application comprising the following steps: inoculating a single colony of the Zygosaccharomyces bailii CCTCC NO: M 2023729 into a sorghum juice medium, and culturing at 30° C. and 200 rpm for 24 hours to obtain a seed culture, and inoculating 1 mL of the seed culture of the Zygosaccharomyces bailii CCTCC NO: M 2023729 into 100 mL of a fermentation medium added with 40 g/L lactic acid, and culturing at 30° C. and 200 rpm for 72 hours; wherein the sorghum juice medium is prepared by soaking sorghum in water at a ratio of 1:4 (M/V) overnight, adding amylase for steaming and liquefaction, adding glucoamylase for saccharification at 60° C., performing filtration and centrifugation, and adjusting a sugar content to 5° Bx.; and the fermentation medium is prepared by adjusting a pH value of the sorghum juice medium to 3.2 using lactic acid.

8. Application of a yeast agent in Baijiu (Chinese liquor) brewing, comprising: inoculating Zygosaccharomyces bailii CCTCC NO: M 2023729 into a seed culture medium to obtain a seed culture; centrifuging the seed culture to remove a supernatant, and resuspending precipitate in water to obtain the yeast agent; wherein
the yeast agent is applied to a fourth fermentation round of a Maotai-flavor liquor brewing process, with steps as follows:
distilling, spreading and cooling a fermented grain, mixing the cooled fermented grain with Qu, a Chinese fermentation starter, taking out the fermented grain, evenly spraying the yeast agent onto the fermented grain, and mixing thoroughly; and placing the mixed fermented grain into a bamboo basket, and placing in a fermentation pile and a fermentation pit for fermentation under in-situ conditions.
